(19) United States
(12) Patent Application Publication
(10) Pub. No.: US 2023/0000065 A1
(43) Pub. Date: Jan. 5, 2023

(54) MITE COMPOSITION AND METHOD FOR REARING MITES

(71) Applicant: BIOBEST GROUP N.V., Westerlo (BE)

(72) Inventors: Dominiek VANGANSBEKE, Westerlo (BE); Marcus Vinicius Alfenas DUARTE, Westerlo (BE); Alfredo BENAVENTE MARTINEZ, Westerlo (BE); Jonas Andree Jozef MERCKX, Westerlo (BE); Manon Helene Lionella GUILBAUD, Westerlo (BE); Keith George OAKLEY, Westerlo (BE); Karel Jozef Florent Bolckmans BOLCKMANS, Westerlo (BE); Felix WACKERS, Westerlo (BE)

(21) Appl. No.: 17/781,934

(22) PCT Filed: Dec. 4, 2020
(86) PCT No.: PCT/EP2020/084674

§ 371 Date: Jun. 2, 2022

(30) Foreign Application Priority Data
Dec. 4, 2019 (BE) 2019/5864

(51) Int. Cl. A01K 67/033; A23K 50/90; A23K 10/30; A01N 63/16

(52) U.S. Cl. CPC A01K 67/033; A23K 50/90; A23K 10/30; A01N 63/16

(57) ABSTRACT

The present invention relates to methods for the mass rearing of predatory mites, mite compositions and the use thereof for biological control. In particular, the present invention relates to the use of prey mites from the genus Czenspinskia for the mass rearing of predatory mites and for controlling a pest in a crop.

### FIELD OF THE INVENTION

The present invention relates to methods for the mass rearing of predatory mites, mite compositions and the use thereof for biological control. In particular, the present invention relates to the use of prey mites from the genus Czenspinskia for the mass rearing of predatory mites and for controlling a pest in a crop.

### BACKGROUND OF THE INVENTION

Predatory mites are traditionally used in the broad field of agricultural pest management. A wide range of predatory mite species have been suggested or commercialized for the biological control of phytophagous pest mites and insect pests such as whiteflies and thrips.

Lately, the major arthropod pest species have mainly been controlled by chemical agents, i.e. pesticides. Nowadays, many of the new pesticides made available on the market are more selective and less hazardous than the older compounds. However, even the newest pesticides present several major problems, i.e. the development of resistance in target pest species; the dwindling supply of useful, registered insecticides and acaricides; the deposition of undesirable residues; the detrimental effect on non-target species resulting in secondary pest outbreaks; the phytotoxic reactions induced in treated plants. It is therefore becoming increasingly clear that solely relying on chemical control will not be the solution to the problem of agricultural pest management. For this reason, many farmers and gardeners are exploring and adopting methods to reduce pesticide use.

One alternative to the use of chemical agents is biological control. Biological control is the intentional manipulation of populations of living beneficial organisms (natural enemies) in order to limit population of pests. Natural enemies of mites for instance include predators, parasitic insects, nematodes, and pathogens. Indeed, virtually all pests have natural enemies and appropriate management of such natural enemies can effectively control many pests.

The objective of biological control is not to eradicate pests, but to maintain them at tolerable levels at which they cause no appreciable damage. As such, biological control can be effective, economical and safe.

Since many years, predatory mite populations have been used in order to control pests. In particular, primarily phytoseiid predatory mites are currently used to control pests such as phytophagous mites, thrips and whiteflies. For example, Neoseiulus cucumeris (Oudemans) is commercially used for the control of thrips larvae and spider mites. Other predatory mites species, particularly mesostigmatid and prostigmatid species, receive particular attention in the context of biological pest control, and some are already commercialized.

There is therefore a growing interest for the development of effective mass rearing systems to produce predatory mite populations on commercially-relevant scales, and this for an acceptable price. Currently, commercial rearing systems use living preys in a culture maintained on a carrier to rear predatory mites. For example, WO2006/071107 and WO2013/103295 disclose a mite composition comprising a population of individuals of a predatory mite species, a prey mite population as a food source for the predatory mite individuals and a carrier. The compositions according to these prior art documents are suitable for rearing mite species and for the biological control of pests. Mass rearing systems for predatory mites depend heavily on the availability of suitable prey for the predators. Considering their role in rearing of predatory mites, the commercial relevance of rearing prey mites is increasing.

WO2006/057552, WO2008/015393, WO2008/104807 and WO2007/075081 demonstrate the potential of Astigmatid mites to be used as prey mites in mass-rearing of predatory mites. However, until now, only a few Astigmatid prey mite species are used in mass rearing methods, i.e. Tyrophagus putrescentiae (Schrank), Thyreophagus entomophagous (Laboulbéne & Robin) and Carpoglyphus lactis L.

Identifying new prey mites for use in biological pest control, which are at the same time economical and easy to breed remains a challenge. Most mites that are preyed upon by predatory mites in nature have subsequently been found not to be suitable for mass-rearing of these predatory mites, for example because they do not reach sufficiently high densities or have defense mechanisms against predatory mites (see e.g. Massaro, M., Martin, J. P. I. and de Moraes G. J. Exp Appl Acarol (2016) 70: 411; Barbosa, M. F. C. and de Moraes, G. J. Exp Appl Acarol (2016) 69: 289; Barbosa, M. F. C. and de Moraes, G. J. Biological Control (2015) 91: 22-26; and Midthassel, A., Leather, S. R., Wright, D. J. et al. BioControl (2016) 61: 437). Furthermore, any potential negative impact of the prey mite on crop plants as well as end users should be minimal.

In view of the above there is a continuing need to obtain improved (more efficient) mite compositions for mass rearing and large-scale production of predatory mite populations, for commercial distribution of larger volumes of mite compositions comprising such and for better pest control and agricultural management.

### SUMMARY OF THE INVENTION

The inventors have surprisingly found that the use of mites from the genus Czenspinskia as prey mites overcome the problems of the prior art.

Therefore the present invention provides a mite composition comprising
a predatory mite population,
a prey mite population, and
a carrier for individuals of said populations,
   wherein said prey mite population comprises mites from the genus Czenspinskia.

### BRIEF DESCRIPTION OF THE DRAWINGS

FIG. 1: Number of eggs deposited by a female A. swirskii for 3 consecutive days when fed on mixed stages of C. transversostriata and C. lactis at 22±1° C. and 85±5% RH (contrasts after GLM, p>0.05).

FIG. 2: Capture success rate of A. swirskii females on adults of C. transversostriata and C. lactis (expressed as proportion of female prey killed). The asterisk indicates a significant difference between the prey mite species (contrasts after GLM, p<0.05).

### DETAILED DESCRIPTION OF THE INVENTION

As described herein before, the present invention provides a mite composition comprising
a predatory mite population,
a prey mite population, and
a carrier for individuals of said populations,
   wherein said prey mite population comprises mites from the genus Czenspinskia.

By the term «predatory mite population», is meant in the sense of the present invention, a population of beneficial mites that feeds on a prey mite population.

By the term «prey mite population», is meant in the sense of the present invention, a population of mites that at least partially consumed by a predatory mite population.

By the term «carrier», is meant in the sense of the present invention, any solid material which is suitable to provide a carrier surface to the individuals of both the predatory and the prey mite populations. The carrier will usually act as a three-dimensional matrix wherein the prey mite population and predatory mite population can move around, hide, develop and prey or be preyed upon.

The genus Czenspinskia has a wide distribution and mites can be readily isolated from nature for use in the present invention. A preferred species is Czenspinskia transversostriata, also known as Calvolia transversostriata or Czenspinskia lordi.

Inventors surprisingly observed that a mite composition according to the invention shows better results in terms of efficiency. It was observed that the mites from the genus Czenspinskia of the prey mite population are more easily killed and more readily eaten by the predatory mite population than the conventionally used astigmatid prey mites. An additional advantage is that all life stages of the Czenspinskia prey mites are being killed readily, whereas larger life stages, such as adults, from other astigmatids are not killed at all or killed at a much lower rate. In addition, it was observed that even at high densities, the prey mite population disturbs the predatory mite population less than the prey mites used in the prior art for mass rearing mites. Although not wishing to be bound by theory, it appears that the Czenspinskia mites produce repellent volatiles to a much lower degree, compared to prior art prey mites. This may cause the Czenspinskia prey mites to have a lower disturbing effect on the predatory mite population. Accordingly, individuals from the predatory mite population may grow faster and therefore mature more quickly which allows for a higher rate of reproduction. Such a higher reproductive rate results in a faster increase in the number of individuals in the predatory mite population. This advantage is beneficial for the rearing of predatory mites and the production of compositions comprising such, as well as for the control of a pest in a crop. It is to be noted that the prey mite population used in the invention may also comprise (or consist of) a population of dead prey mites. The observation that the prey mites of the invention are more readily eaten than the prey mites of the prior art, also applies to dead Czenspinskia mites in comparison to dead prior art prey mites. The use of a prey mite population comprising dead prey mites provides the benefit that predatory mites require less energy for killing and consuming the prey mites. Czenspinskia prey mites have been found to be particularly useful for rearing phytoseiid predatory mites species, such as Amblyseius swirskii, at least as potent as the most commonly used ‘golden standard’ Carpoglyphus lactis L.

Furthermore, Czenspinskia, and especially Czenspinskia transversostriata, has a worldwide distribution. Given the wide distribution in nature, the mite compositions of the invention can furthermore be safely utilized worldwide without a risk to introduce non-native, invasive species. In addition, in contrast to several prey mites that have been used in the prior art for mass rearing of predatory mites, Czenspinskia is neither a storage nor plant pest. As such, it does not pose a potential risk for the environment and the end users. The prey mites of the invention therefore also provide a safer solution for mass rearing of predatory mites.

In a preferred embodiment of the invention, the prey mites are Czenspinskia transversostriata (Oudemans) mites.

Preferably, the Czenspinskia mites according to the invention do not cause plant damage. The skilled person is well aware how to select Czenspinskia mites that do not cause significant plant damage. In addition, the absence of causing significant plant damage is easily determined, e.g. by incubating a plant with the Czenspinskia of choice and determining if the plant is damaged after a few days. In a particular embodiment, the prey mites according to the invention include mites from the genus of the Czenspinskia.

Advantageously, the composition according to the invention comprises a food source for the predatory and/or prey mite populations. In a particular embodiment, the composition of the invention comprises a food source for said prey mite population. In another embodiment, the food source comprises wheat germ, pollen, or fungus, such as Saccharomyces cerevisae. As is known to the skilled person, a carrier can be selected that acts both as a carrier and as a food source, for example wheat germ.

Advantageously, as explained herein before, the composition according to the invention may further comprise a fungus. Better efficiency results were observed when fungi are added to the composition according to the invention. In fact, the prey mite population feeds on fungi. Thus, the active addition of fungi in the composition according to the invention makes it possible to improve the growth and therefore to accelerate the reproduction maturity of Czenspinskia mites, which will lead to a faster increase of Czenspinskia individuals of the prey mite population. In addition, it has been observed that the active addition of beneficial fungi stabilizes the rearing cultures and further suppresses the growth of unwanted fungal contaminations. Accordingly, the active addition of fungi in the composition according to the invention will indirectly enhance the rearing of predator mite population, their large-scale production and the distribution of the composition according to the invention at larger volumes but will also show better effects on pest control on crops. In a preferred embodiment, the fungus is a yeast, preferably a Saccharomycetes, such as from the genus Saccharomyces. In a preferred embodiment, the fungus is Saccharomyces cerevisiae. A supplemental food source as used herein refers to a food source that is present in addition to the prey mite population. Supplemental food sources are sometimes used in predatory mite rearing as a food source that is more cost-effective than prey mites. It is often observed that several predatory mites can use supplemental foods for obtaining additional energy, but also require the presence of prey mites for survival, development and reproduction. A major drawback of the use of a supplemental food source is that it is prone to fungal contamination. By combining a supplemental food source with the prey mites of the invention, this drawback is suppressed. Examples of preferred supplemental foods are natural food sources (such as pollen or dead mites) and artificial foods (Wäckers et al. (2005) Plant-provided food for carnivorous insects: a protective mutualism and its applications 356p; Morales-Ramos et al. (2014) Mass production of beneficial organisms 742p; Cohen (2015) Insect diets. Science and Technology, Second edition, 473p) Supplemental food sources for the rearing of predatory mites are well-known to the skilled person.

As mentioned herein before, the carrier can be any solid material, which is suitable to provide a carrier surface to the individuals. In a preferred embodiment, the carrier is a mixture of solid carrier elements. Preferably, the carrier is a substantially homogeneous mixture of solid carrier elements. While the solid carrier elements may have a variety of sizes, a substantially homogeneous mixture refers to the variety of sizes being substantially homogeneously distributed amongst the carrier.

The average longest axis of the solid carrier elements is typically between 1.0 and 20.0 mm, more in particular between 2.0 and 12.0 mm, preferably between 3.0 and 9.0 mm. In another particular embodiment, the carrier is a mixture of solid carrier elements with a similar size. In a further embodiment, 90% of the solid carrier elements have a longest axis in the range of 0.1 times to 10.0 times the average longest axis of the mixture of solid carrier elements, in particular in the range of 0.2 and 5.0 times, more in particular in the range of 0.5 and 2.0 times. As will be understood from the disclosures herein, two or more different types of carrier elements may be used and mixed. In such case, the size distribution refers to the size distribution per type of carrier elements. Preferably the carrier provides a porous medium, which allows exchanges of metabolic gases and heat produced by the mite populations. Examples of suitable carriers are non-photosynthetic plant material, such as (wheat) bran, buckwheat husks, rice husks, saw dust, corn cob grits, etcetera, or inorganic material, such as vermiculite. Advantageously, said carrier comprises grains of a grass species or any part thereof, such as germ or bran. Preferably, said carrier does not comprise living, green plant material, such as leaves or stems of plants. In a particular embodiment, the carrier comprises non-photosynthetic plant material. Particularly preferred is a carrier selected from vermiculite, wheat bran, millet chaff, rice husks and buckwheat husks.

Preferably, said carrier comprises carrier elements with an average longest axis of between 1.0 and 20.0 mm, in particular between 3.0 and 9.0 mm. The carrier material may provide shelter for the prey mites and predatory mites, reducing stress conditions and cannibalism. During mass rearing, the carrier allows for the generation of a three-dimensional rearing matrix and may provide a food source for the prey mites. During storage and distribution for biological control, the carrier acts as a bulking agent and allows for a more homogeneous distribution of the predatory mites in the crops.

Advantageously, the number of individuals of the predatory mite population relative to the number of individuals of the prey mite population is from about 1:1 to 1:500, such as about 1:10 to 1:250, and preferably between 1:25 and 1:150.

High densities of prey mites and predatory mites can be reached thanks to the present invention. In a particular embodiment, the mite compositions of the present invention comprise at least 1.000 mites of the prey mite population per gram of the composition, in particular at least 2.000 prey mites, more in particular at least 3.000 prey mites. In a further embodiment, at least 5.000 prey mites, at least 10.000 prey mites, at least 20.000 prey mites, at least 30.000 prey mites. In a preferred embodiment, the mite composition comprises at least 50.000 mites of the prey mite population per gram of the composition.

In another particular embodiment, the mite compositions of the present invention comprise at least 20 mites of the predatory mite population per gram of the composition, in particular at least 30 predatory mites, more in particular at least 50 predatory mites. In a further embodiment, at least 100 predatory mites, at least 150 predatory mites, at least 200 predatory mites, at least 300 predatory mites. In a preferred embodiment, the mite composition comprises at least 500 mites of the predatory mite population per gram of the composition.

Mite numbers are mentioned herein refer to the total number of all mite development stages, thus including eggs, larvae, nymphs and adults, unless the context clearly dictates otherwise. As will be understood from the disclosures herein, the prey mites in the compositions of the invention are not required to be living prey mites.

In a particular embodiment, the mite compositions of the present invention comprise at least 1.000 non-egg mites of the prey mite population per gram of the composition, in particular at least 2.000 non-egg prey mites, more in particular at least 3.000 non-egg prey mites. In a further embodiment, at least 5.000 prey non-egg mites, at least 10.000 prey non-egg mites, at least 20.000 prey non-egg mites, at least 30.000 prey non-egg mites. In a preferred embodiment, the mite composition comprises at least 50.000 non-egg mites of the prey mite population per gram of the composition.

In another particular embodiment, the mite compositions of the present invention comprise at least 20 non-egg mites of the predatory mite population per gram of the composition, in particular at least 30 non-egg predatory mites, more in particular at least non-egg 50 predatory mites. In a further embodiment, at least 100 non-egg predatory mites, at least 150 non-egg predatory mites, at least 200 non-egg predatory mites, at least 300 non-egg predatory mites. In a preferred embodiment, the mite composition comprises at least 500 non-egg mites of the predatory mite population per gram of the composition.

In another particular embodiment, the mite compositions of the present invention comprise at least 20 adult mites of the predatory mite population per gram of the composition, in particular at least 30 adult predatory mites, more in particular at least 50 adult predatory mites. In a further embodiment, at least 100 adult predatory mites, at least 150 adult predatory mites, at least 200 adult predatory mites, at least 300 adult predatory mites. In a preferred embodiment, the mite composition comprises at least 500 adult mites of the predatory mite population per gram of the composition. In further embodiment, the mite compositions of the present invention comprise at least 20 living adult mites of the predatory mite population per gram of the composition, in particular at least 30 living adult predatory mites, more in particular at least 50 living adult predatory mites. In a further embodiment, at least 100 living adult predatory mites, at least 150 living adult predatory mites, at least 200 living adult predatory mites, at least 300 living adult predatory mites. In a preferred embodiment, the mite composition comprises at least 500 living adult mites of the predatory mite population per gram of the composition.

Evidently, the above ranges of prey mite and predatory mite numbers can be combined to arrive to particular and preferred embodiments of the invention. It thus follows that the present invention provides, for example, mite compositions comprising at least 1.000 mites of the prey mite population and at least 20 mites of the predatory mite population per gram of the composition. As another example, at least 5.000 mites of the prey mite population and at least 100 mites of the predatory mite population. As another example, at least 50.000 mites of the prey mite population and at least 500 mites of the predatory mite population.

In a preferred embodiment, said predatory mites are mesostigmatid or prostigmatid mite species. In a further embodiment, said predatory mites are selected from:
Mesostigmatid predatory mite species such as:
   i) Phytoseiidae such as from:
   a)—the subfamily of the Amblyseiinae, such as from the genus Amblyseius, e.g. Amblyseius andersoni, Amblyseius aerialis, Amblyseius swirskii, Amblyseius herbicolus or Amblyseius largoensis, from the genus Euseius e.g. Euseius finlandicus, Euseius hibisci, Euseius ovalis, Euseius victoriensis, Euseius stipulatus, Euseius scutalis, Euseius tularensis, Euseius addoensis, Euseius concordis, Euseius ho or Euseius citri, from the genus Neoseiulus e.g. Neoseiulus barkeri, Neoseiulus califomicus, Neoseiulus cucumeris, Neoseiulus longispinosus, Neoseiulus womersleyi, Neoseiulus idaeus, Neoseiulus anonymus, Neoseiulus paspalivorus, Neoseiulus reductus or Neoseiulus fallacis, from the genus Amblydromalus e.g. Amblydromalus limonicus from the genus Typhlodromalus e.g. Typhlodromalus aripo, Typhlodromalus laila or Typhlodromalus peregrinus from the genus Transeius e.g. Transeius montdorensis, from the genus Phytoseiulus, e.g. Phytoseiulus persimilis, Phytoseiulus macropilis, Phytoseiulus longipes, Phytoseiulus fragariae;
   b) the subfamily of the Typhlodrominae, such as from the genus Galendromus e.g. Galendromus occidentalism from the genus Typhlodromus e.g. Typhlodromus gyri, Typhlodromus doreenae, Typhlodromus rhenanus or Typhlodromus athiasae;
   c) the subfamily of the Phytoseiinae, such as from the genus Phytoseius e.g. Phytoseius macropilis, Phytososeius finitimus or Phytoseius plumifer.
   ii) Ascidae such as from the genus Proctolaelaps, such as Proctolaelaps pygmaeus (Muller); from the genus Blattisocius e.g. Blattisocius tarsalis (Berlese), Blattisocius keegani (Fox); from the genus Lasioseius e.g. Lasioseius fimetorum Karg, Lasioseius floridensis Berlese, Lasioseius bispinosusEvans, Lasioseius dentatus Fox, Lasioseius scapulatus (Kenett), Lasioseius athiasae Nawar & Nasr; from the genus Arctoseius e.g. Arctoseius semiscissus (Berlese); from the genus Protogamasellus e.g. Protogamasellus dioscorus Manson;
   iii) Laelapidae such as from the genus Stratiolaelaps e.g. Stratiolaelaps scimitus (Womersley) (also placed in the genus Hypoaspis) ; Gaeolaelaps e.g. Gaeolaelaps aculeifer (Canestrini) (also placed in the genus Hypoaspis); Androlaelaps e.g. Androlaelaps casalis (Berlese);
   iv) Macrochelidae such as from the genus Macrocheles e.g. Macrocheles robustulus (Berlese), Macrocheles muscaedomesticae (Scopoli), Macrocheles matrius (Hull);
   v) Parasitidae such as from the genus Pergamasus; e.g. Pergamasus quisquiliarum Canestrini; Parasitus e.g. Parasitusfimetorum (Berlese), Parasitus bituberosus Karg; Parasitellus e.g. Parasitellus fucorum (De Geer);
   vi) Digamasellidae such as from the genus Digamasellus; e.g. Digamasellus quadrisetus or Digamasellus punctum; from the genus Dendrolaelaps; e.g. Dendrolaelaps neodisetus; and
   prostigmatid mite species such as from:
   i) Tydeidae such as from the genus Homeopronematus e.g. Homeopronematus anconai (Baker); from the genus Tydeus e.g. Tydeus Iambi (Baker), Tydeus caudatus (Duges); from the genus Pronematus e.g. Pronematus ubiquitus (McGregor);
   ii) Cheyletidae such as from the genus Cheyletus e.g. Cheyletus eruditus (Schrank), Cheyletus malaccensis Oudemans;
   iii) Cunaxidae such as from the genus Coleoscirus e.g. Coleoscirus simplex (Ewing), from the genus Cunaxa e.g. Cunaxa setirostris (Rermann);
   iv) Erythraeidae such as from the genus Balaustium e.g. Balaustium putmani Smiley, Balaustium medicagoense Meyer & Ryke, Balaustium murorum (Hermann);
   v) Stigmaeidae such as from the genus Agistemus e.g. Agistemus exsertus Gonzalez; such as from the genus Zetzellia e.g. Zetzellia mali (Ewing); and
   vi) Tarsonemidae such as from the genus Acaronemus, e.g. Acaronemus destructor (Smiley and Landwehr); such as from the genus Dendroptus near suski Sharonov and Livshits; such as Lupotarsonemus floridanus Attiah.

In a preferred embodiment, the predatory mites belong to the Phytoseiidae. In a particular embodiment, the predatory mites are selected from Amblyseiinae, Transeius, Neoseiulus and Amblydromalus; more in particular the predatory mites are selected from the group consisting of Amblyseius swirskii, Transeius montdorensis, Neoseiulus californicus and Amblydromalus limonicus.

The present invention also provides a commercial packaging for storing and distributing the composition of the invention. Therefore, the present invention further pertains to a container comprising a composition according to the invention. In particular, the container has an internal volume of between 0.2 I and 3 I, preferably between 0.5 I and 2 I. According to a preferred embodiment the container preferably comprising an exit for at least one motile life stage of the mite, more preferably an exit suitable for providing a sustained release of said at least one motile life stage. In a particular embodiment, the container (e.g. a bottle or a sachet) has at least one exit with a removable seal. In particular, seal as used herein refers to a closure that is glued or heat-sealed. Preferably, the container is adapted to attach it to a crop, for example by comprising a hook to hang it from a crop leaf or branch or by comprising a sticky surface to stick it to a surface of the crop.

The present invention further pertains to a method for rearing predatory mites, the method comprising
providing a composition of the invention, and
allowing individuals of the predatory mite population to feed on said prey mite population.

Preferably, the individuals of the predatory mite population are allowed to feed on said prey mite population while maintaining said mite composition at 5 to 35° C. and 30 to 100% relative humidity; in particular at 15 to 35° C. and 50 to 100% relative humidity.

The present invention further pertains to a method for controlling a pest in a crop, the method comprising providing to said crop a mite composition according to the invention. The mite composition of the invention may be distributed directly onto the crop. Alternatively, the mite composition of the invention is provided in the proximity of the crop. For example, the mite composition of the invention may be provided to a crop by placing a container holding the mite composition in the vicinity of the crop and allowing the predatory mites to exit from said container. This way, predatory mites spread themselves through the crops and control the crop pests. In a preferred embodiment, the pest is an arthropod pest.

These and other embodiments of the invention are indicated in the appended claims. The invention will now be further described with reference to the following examples, which show non-limiting embodiments of different aspects of the invention.

### EXAMPLES

### Example 1: Oviposition Rate of Amblyseius swirskii on Different Prey Mites.

This first example was conducted to evaluate the nutritional quality of a Czenspinskia prey mite as compared to a standard used astigmatid prey mite for a phytoseiid predatory mite. More specifically, the oviposition rate of the predatory mite Amblyseius swirskii Athias-Henriot (Acari: Mesostigmata: Phytoseiidae) was tested on the Czenspinskia prey mite Czenspinskia transversostriata (Oudemans) (Acari: Astigmata: Winterschmidtiidae) as compared to a carpoglyphid prey mite, namely Carpoglyphus lactis L. (CL) (Acari: Astigmata: Carpoglyphidae) such as disclosed for A. swirskii rearing in WO2006/071107.

Prey mites were cultured on a medium containing bran, wheat germ and yeast in plastic containers (8×5,7 cm) with a ventilated lid. The containers were maintained at a temperature of 22±1° C. and a relative humidity of 85±5%.

A single gravid female A. swirskii was transferred from mass-rearing facilities onto a black PVC arena (2×4 cm) placed on a layer of wet cotton. The edges of the arena were covered with tissue paper to prevent mites from escaping. A black piece of plastic (1×1 cm) with cotton threads was added to provide shelter and oviposition substrates. Prey mites were provided ad libitum. The number of eggs deposited by each female was counted daily during 4 consecutive days. The oviposition rate of the first day was omitted from analysis to limit the effect of the diet prior to the experiment (Sabelis 1990). The sum of the amount of eggs laid during day 2, 3 and 4 were compared with GLM model with Poisson distribution. Contrasts among treatments were assessed by stepwise model simplification through aggregation of nonsignificant factor levels (Crawley 2013). All analyses were performed using the statistical software R 3.10 (RDevelopment Core Team 2012).

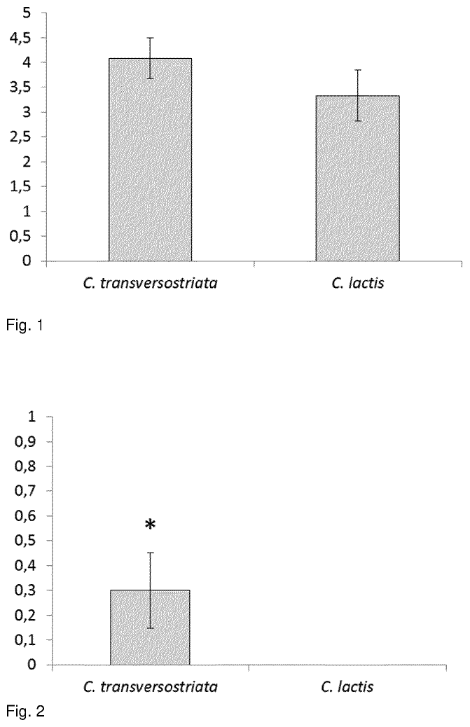

The results demonstrate that A. swirskii is able to reproduce on the Czenspinskia prey mite C. transversostriata. The oviposition rate observed for Czenspinskia was higher than for A. swirskii, although it did not reach statistical significance in this experiment in the egg laying rate between missed stages of either prey species ((GLM, X₂=0.91167, d.f.=1, p=0.3397) (FIG. 1). Similar amounts of eggs were laid when grown on C. transversostriata prey mites or C. lactis prey mites. In conclusion, Czenspinskia mites are a suitable prey mite population for the commercial rearing of predatory mites, such as A. swirskii.

### Example 2: Capture Success Rate of A. swirskii When Provided Adult Stages of Different Prey Mites.

Previous studies have reported that adult stages of most astigmatid prey mites are more difficultly subdued and consumed than eggs and larvae of these mites. In the present example, the capture success rate was assessed for the adult stages of the Czenspinskia prey mite C. transversostriata, and the carpoglyphid prey mite C. lactis.

Predatory and prey mites were reared as described in example 1. Female A. swirskii were starved for 16h prior to start of the experiment by transferring them to a black PVC plate without any food source. After 16 h, a single female A. swirskii was transferred to a black PVC arena as described in experiment 1. Prior to the introduction of the A. swirskii female, 25 adults of the two prey mites were transferred to the experimental arena. After introduction of the predator, observations were done for 5 minutes or until a successful attack (i.e. killing and feeding on the prey). If a female did not succeed in killing prey within 5 minutes, this was recorded as unsuccessful. For each treatment, 10 replicates were set up. Data were compared among treatments with a GLM model with binomial distribution. All statistical analyses were done using the computer software R version 3.1.0 (R Core Team 2014).

When adult Czenspinskia females were provided, a high proportion of the prey individuals were subdued as compared to the carpoglyphid species where none of the prey mites were successfully subdued (GLM, _{X}2=4.6911, d.f.=1, p=0.0303).

From the results presented in FIG. 2, it is clear that A. swirskii females are able to kill and consume much more of the adults of the Czenspinskia prey mite C. transversostriata as compared to the conventionally used astigmatid prey mite species. Thus, for Czenspinskia prey mites, adult stages are more easily subdued and killed, making it a more suitable prey.

1. A mite composition comprising:
a predatory mite population,
a prey mite population, and
a carrier for individuals of said populations,
wherein said prey mite population comprises mites from the genus Czenspinskia.

2. The composition according to claim 1, further comprising a food source for said mite populations.

3. The composition according to claim 1, wherein the food source for said prey mite population comprises wheat germ; pollen, or a fungus, such as yeast.

4. The composition of claim 1, wherein said carrier comprises grains of a grass species or any part thereof, such as germ or bran.

5. The composition of claim 1, wherein said carrier comprises carrier elements with an average longest axis of between 1.0 and 20.0 mm.

6. The composition of claim 1, wherein the number of individuals of the predatory mite species relative to the number of individuals of the prey mite is from about 1:1 to about 1:500; in particular from about 1:50 to about 1:200; more in particular between 1:75 and 1:125.

7. The composition of claim 1, wherein said prey mite population comprises mites from the species Czenspinskia transversostriata.

8. The composition of claim 1, wherein said predatory mite species are mesostigmatid or prostigmatid mite species, in particular Phytoseiidae.

9. The composition of claim 1, wherein the composition comprises at least 200 prey mites of the genus Czenspinskia per gram of the composition and at least 10 predatory mites per gram of the composition.

10. A container comprising the composition of claim 1.

11. A method for rearing predatory mites, the method comprising
providing the composition of claim 1, and
allowing individuals of the predatory mite population to feed on said prey mite population.

12. The method of claim 11, further comprising maintaining said mite composition at 5 to 35° C. and 30 to 100% relative humidity.

13. The method of claim 12, further comprising packaging the composition in a container with an exit that is designed to be opened to release at least individuals from the predatory mite population from the container.

14. A method for controlling a pest in a crop, the method comprising providing to said crop a mite composition according to claim 1.

15. The method of claim 14, comprising placing the container of claim 10 in or at close proximity to the crop.
